(19) United States
(12) Patent Application Publication
(10) Pub. No.: US 2010/0030159 A1
(43) Pub. Date: Feb. 4, 2010

(54) SYRINGE PLUNGER

(76) Inventor: Jian-Xin Li, Guangdong (CN)

Correspondence Address: Muncy, Geissler, Olds & Lowe, PLLC, P.O. BOX 1364, FAIRFAX, VA, 22038-1364, US

(21) Appl. No.: 12/524,978

(22) PCT Filed: Nov. 19, 2007
(86) PCT No.: PCT/CN07/03267

§ 371 Date: Jul. 29, 2009

(30) Foreign Application Priority Data
Jun. 11, 2007 (CN) 200710028512.9

(51) Int. Cl. A61M 5/315; B29C 45/14

(52) U.S. Cl. 604/218; 264/271.1

(57) ABSTRACT

A syringe plunger has a hole is arranged in its plunger central axis, with a diaphragm arranged in the hole. The thickness of the diaphragm in the hole is from 0.01 mm to 0.5 mm. A manufacturing method of a syringe plunger includes the steps of: adding pigment into medical rubber and stirring uniformly; each of a fixed die and a movable die of a medical rubber syringe die being embedded with an impacting and penetrating needle, when molding and die closing, space between the impacting and penetrating needles of the fixed die and the movable die being 0.01 mm to 0.5 mm; demolding said molded medical rubber plunger.

### BACKGROUND OF THE INVENTION

1. Field of the Invention

The present invention relates to a syringe device, and in particular to a syringe plunger and a manufacturing method of the same.

2. The Prior Arts

Nowadays, a syringe is a device/instrument utilized frequently in curing diseases of people, since it provides a very fast and convenient means for injecting various medicines into human bodies, as such, it is widely utilized in the medical profession in curing patients for its various advantages of low cost, high safety, and easy manufacturing.

However, in an ejection and molding process of a plunger utilized in a syringe, on the end surface of a plunger having holes, a thin layer of flash usually appears, such that in applying a plunger in a syringe, and when the syringe takes in a liquid medicine, these tiny flashes on the end surface of a plunger can be dissolved easily into the liquid medicine, thus affecting the effectiveness of curing a disease by the medicine, or even worse, it may produce harmful side effects to human body. Due to the high concerns and expectations of the public about the health problem, this kind of shortcomings and deficiencies can not be allowed to happen. Therefore, it is a serious problem that must be urgently solved in this field.

### SUMMARY OF THE INVENTION

In order to solve the above-mentioned shortcomings and drawbacks of the prior art, the present invention discloses a syringe plunger and method of manufacturing the same, so as to solve the problem of the prior art that the flashes on the end surface of a medical rubber plunger tend to produce tiny burrs, and that will adversely affect the health of a human body.

To achieve the above-mentioned objective, the present invention provides a syringe plunger, and it is characterized in that the syringe plunger has a hole arranged in its plunger central axis, with a diaphragm arranged in the hole, and the thickness of the diaphragm in the hole is from 0.01 mm to 0.5 mm.

In addition, the present invention also provides a method of manufacturing a syringe plunger, including the following steps of: adding pigment into medical rubber (including various medical rubbers, solid silicon, liquid silicon, etc.) and stirring the mixture uniformly; each of a fixed die and a movable die of medical rubber syringe die is embedded with an impacting and penetrating needle, when molding and die closing, space between the impacting and penetrating needles of the fixed die and the movable die is 0.01 mm to 0.5 mm, the important point is that the die are designed to have the impacting and penetrating needles for the holes in the plunger, yet, when molding, the impacting and penetrating needles actually do not impact and penetrate each other; demolding the molded medical rubber plunger; placing the demolded plunger into a sulfurator for sulfurization processing; placing the demolded plunger into a flash nitration processing unit for nitration processing, the sulfurization processing mentioned above is conducted in a temperature of 200° C. for four hours, and nitration processing is conducted in a temperature of −160° C. for 10 to 20 minutes.

From the above description it can be known that, in the present invention, syringe plunger is made of liquid silicon, since liquid silicon is an environment friendly material, having no toxic or harmful side effects to human body, besides, the chemical property of liquid silicon is rather stable, and it will not produce chemical reactions with the liquid medicine inside the syringe, thus it can be utilized safely. In addition, with regard to the ejection and molding technology of a plunger, in the present invention, a two-needle-injection-hole method is adopted in keeping a specific distance between two needles, hereby avoiding producing flashes on two end surfaces of a plunger, thus burrs will not be generated in affecting the health of people.

Further scope of the applicability of the present invention will become apparent from the detailed description given hereinafter. However, it should be understood that the detailed description and specific examples, while indicating preferred embodiments of the present invention, are given by way of illustration only, since various changes and modifications within the spirit and scope of the present invention will become apparent to those skilled in the art from this detailed description.

### BRIEF DESCRIPTION OF THE DRAWINGS

The related drawings in connection with the detailed description of the present invention to be made later are described briefly as follows, in which:

FIG. 1 is a longitudinal cross section view of a syringe plunger according to an embodiment of the present invention; and

FIG. 2 is a horizontal cross section view of a syringe plunger according to an embodiment of the present invention.

### DETAILED DESCRIPTION OF THE PREFERRED EMBODIMENT

The purpose, construction, features, functions and advantages of the present invention can be appreciated and understood more thoroughly through the following detailed description with reference to the attached drawings.

In the present invention, a syringe plunger and a method of manufacturing the same is disclosed. Wherein, a two-needle-penetrating-hole method is adopted in keeping a specific distance between two needles, hereby avoiding producing flashes on two end surfaces of a plunger, thus burrs will not be generated in affecting the health of people.

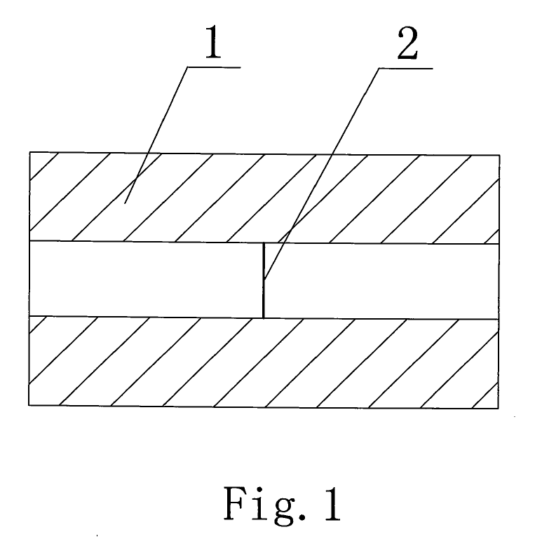

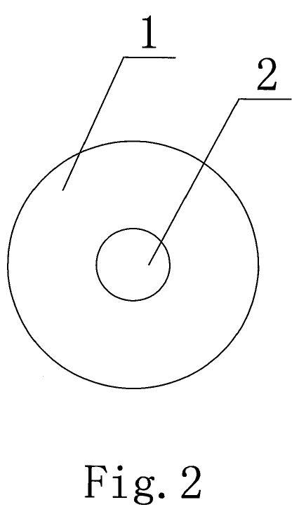

Firstly, refer to FIG. 1 for a longitudinal cross section view of a syringe plunger according to an embodiment of the present invention; and also refer to FIG. 2 for a horizontal cross section view of a syringe plunger according to an embodiment of the present invention. As shown in FIGS. 1 & 2, in the molding process of medical rubber, each of two impacting and penetrating needles are embedded in a fixed die and a movable die respectively, and when molding and dies closing, a spacing exists between the two needles, thus forming a layer of un-penetrated diaphragm of its thickness of about 0.01 mm-0.5 mm, preferably between 0.01 mm and 0.5 mm. When assembling a syringe, a plastic medicine-feeding rod in the syringe is used to penetrate the diaphragm through the hole on the plunger, thus achieving the functions of penetrating the hole and preventing the occurrence of tiny flashes.

The syringe plunger is made of medical rubber by means of ejection molding through the following steps:

adding pigments into medical rubber and stirring the mixture uniformly, namely giving color to the material;

placing and feeding a mixture thus obtained into a silicon ejection molding machine in proceed with ejection molding. In order to make a mold thus produced to be more precise, a cold-glue-chute three-plate type high precision die is adopted for use in ejection molding;

demolding the molded rubber plunger using a robot arm, such that the molded plunger will be demolded automatically;

a blank thus obtained is preferably placed into a sulfurator in proceeding with sulfurization, for increasing the rigidity of the plunger;

a blank thus obtained is preferably placed into a nitrator in proceeding with nitration, for the purpose of deburring and raising the precision of the plunger, so that it may contact the syringe more closely in achieving better hermeticity;

the sulfurization is preferably performed in a temperature of 200° C. for four hours, and the nitration is preferably performed in a temperature of −160° C. for 10-20 minutes.

In order to obtain a clean and non-toxic plunger, the above production steps are conducted in a dustless production room.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A syringe plunger, comprising:
a hole arranged in its plunger central axis, with a diagram arranged in said hole.

2. The syringe plunger as claimed in claim 1, wherein
a thickness of said diaphragm in said hole is from 0.01 mm to 0.5 mm.

3. A syringe plunger manufacturing method, comprising the following steps:
adding pigment into medical rubber and stirring a mixture uniformly,
placing and feeding said mixture thus obtained into a silicon ejection molding machine in proceed with ejection molding, each of a fixed die and a movable die of a medical rubber syringe die is embedded with an impacting and penetrating needle, when molding and die closing, a spacing between said impacting and penetrating needles of said fixed die and said movable die is 0.01 mm to 0.5 mm; and
demolding said molded medical rubber plunger.

4. The syringe plunger manufacturing method as claimed in claim 3, wherein
said demolded plunger is placed into a sulfurator in proceeding with sulfurization; and
said demolded plunger is placed into a nitrator in proceeding with nitration.

5. The syringe plunger manufacturing method as claimed in claim 4, wherein
said sulfurization is performed in a temperature of 200° C. for 4 hours, and said nitration is performed in a temperature of −160° C. for 10-20 minutes.
